(19) United States
(12) Patent Application Publication
(10) Pub. No.: US 2007/0231288 A1
(43) Pub. Date: Oct. 4, 2007

(54) COSMETIC USE OF D-RIBOSE AND METHOD THEREOF

(75) Inventors: Jean-Pierre Arnaud, Verrieres-Le-Buisson (FR); Magali Borel, Paris (FR)

Correspondence Address: MARSH, FISCHMANN & BREYFOGLE LLP, 3151 SOUTH VAUGHN WAY, SUITE 411, AURORA, CO, 80014, US

(73) Assignee: LM Cosmetics, Champlan (FR)

(21) Appl. No.: 11/690,707

(22) Filed: Mar. 23, 2007

(30) Foreign Application Priority Data
Mar. 28, 2006 (FR) 06 02675

(51) Int. Cl. A61K 8/73; A61K 31/70

(52) U.S. Cl. 424/70.13; 514/23

(57) ABSTRACT

The invention relates to the use of D-ribose, in a cosmetic composition intended to optimize the metabolism of skin cells, to alleviate or to prevent the appearance of wrinkles and fine lines, to allow skin elasticity to be improved and to allow the freshness of the skin to be improved and method thereof.

This invention relates to the use of D-ribose, in a cosmetic composition intended to optimise the metabolism of skin cells and method thereof. This novel use has as its object, in particular, the improvement of cellular respiration and the regulation of the production of intracellular ATP in order to optimise the metabolism of the cutaneous cells and tissues.

In fact it is known that ATP plays an essential role in the functioning of each cell. The quantity of ATP produced is very low in relation to the needs of the cell. The molecules of ATP are therefore constantly recycled in order to supply the necessary energy.

When they are subject to different stresses (pollution, tobacco, physical effort, aging, etc.), the cells use more energy than is recycled. The ATP level then reduces and the cellular mechanism is affected by this.

D-ribose is a pentose present in all living organisms. It is an essential element of the structure of the ATP molecule; it is directly involved in de novo ATP synthesis.

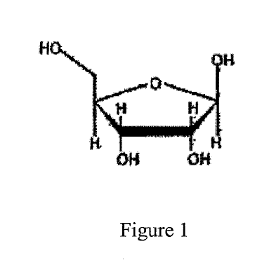

D-ribose is described according to FIG. 1.

The inventors have observed that the incorporation of D-ribose in a cosmetic composition allows in particular, improvement in cellular respiration and de novo intracellular ATP production.

More particularly, the inventors have measured in vitro the effect of D-ribose on cellular energy metabolism, i.e. on the consumption of oxygen and intracellular ATP concentration. They have thus demonstrated that D-ribose acts by increasing the oxygen consumption (mitochondrial and cytosolic respiration) and by regulating the production of ATP by the cells, and have concluded that it improves the metabolism of the cutaneous cells and tissues. The results of these experiments are shown in Examples 1 and 2.

The effect of ribose on the skin has also been evaluated in vivo, by profilometric measurements of wrinkles and fine lines, by DTM measurement of the skin elasticity and by self-assessment by a panel of volunteers. The results are presented in Examples 3, 4 and 5.

The invention thus relates a method to alleviate or to prevent the appearance of wrinkles and fine lines comprising the administration of D-ribose.

More particularly, the subject of the invention relates a method in which D-ribose allows skin elasticity to be improved and in which the D-ribose allows the freshness of the skin to be improved.

According to the method of the invention the D-ribose has a degree of purity of at least 98% (HPLC) and is present in a cosmetic composition at a concentration of 0.01 to 10% by weight with respect to the total weight of the cosmetic composition and preferably is present in a cosmetic composition at a concentration of 0.1 to 1.5% by weight with respect to the total weight of the cosmetic composition.

The invention relates also to the use of D-ribose, in a cosmetic composition intended to optimize the metabolism of skin cells.

More particularly, the subject of the invention is the cosmetic use of D-ribose for optimizing skin cell metabolism and in particular the use of D-ribose in a cosmetic composition as a single active ingredient for optimising the metabolism of the skin cells.

According to the invention, this use of D-ribose in cosmetic compositions is carried out by topical use.

The D-ribose administered in topical form stimulates cellular respiration and de novo ATP production.

Used in this way in a cosmetic composition, D-ribose allows the onset of wrinkles and fine lines to be alleviated or avoided and the elasticity and freshness of the skin to be improved.

According to the invention, the D-ribose is used as pure as possible, preferably with a level of purity of at least 98% (HPLC).

Preferably, the D-ribose is present in a cosmetic composition as the single active ingredient, to improve the metabolism of skin cells.

According to the invention, the D-ribose can be used as it is, in solution, vectorized, microencapsulated, or in combination with a mixture of excipients such as: vegetable oils, mineral oils, vegetable or mineral waxes, silicones, alcohols and fatty acids, surface active agents.

The cosmetic compositions for topical use according to the invention can be formulated in different ways, such as simple emulsions, O/W and W/O emulsions, multiple emulsions or microemulsions, aqueous or lipophilic gels, oils, sprays, sticks, aerosols, toilet products (shampoos, shower gels, etc.).

These compositions are intended for use on the face, body, scalp and hair.

D-ribose can be used together with other active ingredients such as the following products: anti-aging, slimming, seboregulating, moisturizing. Preferably, the cosmetic compositions according to the invention contain from 0.01 to 10%, preferably from 0.1 to 5% of D-ribose by weight of the composition, preferably also from 0.1 to 1.5% by weight with respect to the total weight of the cosmetic composition.

These compositions are prepared according to methods known to a person skilled in the art.

The following examples illustrate the invention without however limiting it.

### EXAMPLES

In the examples, D-ribose obtained by biotechnology was used, with a purity exceeding 98% (HPLC).

### Example 1

Effect of D-Ribose on In Vitro Skin Cell Respiration.

The inventors measured the effect of D-ribose on the mitochondrial and cytosolic respiration of normal human dermal fibroblasts in culture (NHDF).

Method:

The cells used are fibroblasts cultured at 37° C., 5% CO2, in DMEM medium, 10% FCS (foetal calf serum).

The NHDF cultured in monolayer are placed in suspension and distributed in the wells of a special plate of the “BD Oxygen Biosensor System” kit. The bottom of the well is constituted by a membrane on which a molecule is grafted the fluorescence of which is inhibited by the oxygen dissolved in the incubation medium. The fluorescence increases in proportion to the oxygen consumed by the cells. 0.05% D-ribose is added in six wells. The intensity of fluorescence is measured in kinetic mode. The rate of respiration is calculated from measurements of the intensity of fluorescence.

Results:

After 2 hours of contact, an increase in the mitochondrial respiration of 31% and the cytosolic respiration of 37% are noted, at a concentration of D-ribose of 0.05%.

The D-ribose therefore allows the oxygen consumption by the cells to be increased and consequently an increase in the metabolism of the cells and skin tissues.

### Example 2

Effect of D-Ribose on the In Vitro Production of ATP by the Skin Cells.

The inventors measured the effect of D-ribose on the production of ATP by the normal human epidermal fibroblasts in culture, under conditions of hypoxia.

Method:

The cellules used are flask inoculations of normal human epidermal fibroblasts in DMEM medium with 10% FCS, incubation at 37° C., 5% CO2 and RH>95% up to 80% confluency. On Day 1, a first measurement of the level of ATP is taken on the untreated, unstressed NHDF. Pre-treatment for 24 hours with 0.05% of ribose versus an untreated control condition, 2 series in hexaplicate according to conditions. On Day 2, before the induction of hypoxia: stopping a series in order to measure the ATP level before hypoxia over all conditions (treated and untreated). The cells of the 2nd series were placed in suspension, the culture medium was renewed by DMEM 10% FCS supplemented with Hepes buffer (to maintain the pH at physiological conditions during hypoxia) and with EDTA (to prevent cell agglutination). The cells are placed in a incubator adjusted to 37° C., RH>95% the atmosphere of which has previously been replaced with nitrogen. Incubation for 6 hours under conditions of severe hypoxia (oxygen <2%) in the presence of 0.05% D-ribose versus an untreated control condition. Recovery of the cells and measurement of the quantity of ATP in the treated and untreated cells.

Results:

After 6 hours of hypoxia, the presence of 0.05% D-ribose makes it possible for an ATP level of 19% to be re-established.

Under extreme conditions, the D-ribose allows the ATP level to be partially re-established.

### Example 3

Effect of D-Ribose on Skin Elasticity Measured In Vivo.

The inventors showed the effect of ribose on skin elasticity using DTM.

Method:

The measurements of skin elasticity were carried out using a twistometer before the treatment and after 28 days of treatment, at the position of the cheekbones.

A group of 20 women volunteers applied a cosmetic product twice a day containing 0.5% D-ribose (Example 6 Table 1) to the whole face.

Results:

After 28 days of treatment, a significant variation was noted in the values in comparison with Day 0. The cosmetic product containing 0.5% D-ribose allows an average increase of 12.3% in skin elasticity.

### Example 4

Effect of D-Ribose on the Wrinkles Measured In Vivo

The inventors showed the effect of D-ribose on wrinkles using profilometry.

Method:

Impressions of the wrinkles were taken at the position of the crow's foot before the treatment, after 14 days of treatment and after 28 days of treatment.

A group of 20 volunteer women applied a cosmetic product twice a day containing 0.5% D-ribose (formula Example 6 Table 1) to the whole of the face.

Results:

After 14 days of treatment, a significant variation was noted in the values for the treated area in comparison with Day 0. The cosmetic product containing 0.5% D-ribose allows an average reduction of 9.1% in the total length of the wrinkles and 12.2% in the total surface area of the wrinkles.

After 28 days of treatment, a significant variation was noted in the values for the treated area in comparison with Day 0. The cosmetic product containing 0.5% D-ribose allows an average reduction of 17.6% in the average length of the wrinkles and 12.2% in the total surface area of the wrinkles.

### Example 5

Self-Assessment of the Panelists on the Freshness of Complexion

A group of 20 volunteer women applied a cosmetic product twice a day for 28 days containing 0.5% D-ribose (formula Example 6 Table 1) to the whole of the face.

According to the evaluation questionnaire completed by these volunteers, 67% found their skin more radiant/more glowing, 71% that their complexion was less dull, 71% that their complexion was more even.

### Example 6

Useful Cosmetic Compositions According to the Invention.

The percentages are given with respect to the total weight of the composition.

TABLE 1
Anti-wrinkle face emulsion.

| | |
|---|---|
| D-Ribose | 0.5% |
| Chelating agent | 0.10 |
| Emulsifiers | 5 |
| Antioxidant | 0.05 |
| Isononyl isonanoate | 6 |
| Stearyl heptanoate | 3 |
| Silicones | 6 |
| Preservative | 0.80 |
| Perfume | 0.25 |
| Demineralised water | sqf 100 g |

TABLE 2
Oxygenating mask for the face

| | |
|---|---|
| D-Ribose | 1% |
| Glycerine | 4 |
| Chelating agent | 0.10 |
| Emulsifier | 4 |
| Thickener | 0.30 |
| Vitamin B | 0.30 |
| Vegetable oils | 12 |
| Soya isoflavones | 1 |
| Silicones | 9 |
| Anti-microbial agent | 2 |
| Perfume | sq |
| Demineralised water | sqf 100 g |

TABLE 3
Body firming cream.

| | |
|---|---|
| D-Ribose | 1% |
| Glycerine | 2 |
| Chelating agent | 0.10 |
| Emulsifier | 7 |
| Gelling agent | 0.3 |
| Vegetable oils | 3 |
| Vegetal tensor | 2 |
| Silicones | 7.5 |
| Anti-microbial agent | 4.8 |
| Perfume | sq |
| Demineralised water | sqf 100 g |

These compositions were prepared according to known methods by mixing the various ingredients.

1. Method to alleviate or to prevent the appearance of wrinkles and fine lines comprising the administration of D-ribose.

2. Method according to claim 1, in which D-ribose allows skin elasticity to be improved.

3. Method according to claim 1, in which the D-ribose allows the freshness of the skin to be improved.

4. Method according to claim 1, in which the D-ribose has a degree of purity of at least 98% (HPLC).

5. Method according to claim 1 in which the D-ribose is present in a cosmetic composition at a concentration of 0.01 to 10% by weight with respect to the total weight of the cosmetic composition.

6. Method according to claim 1, in which the D-ribose is present in a cosmetic composition at a concentration of 0.1 to 1.5% by weight with respect to the total weight of the cosmetic composition.

7. Use of D-ribose, in a cosmetic composition intended to alleviate or to prevent the appearance of wrinkles and fine lines.

8. Use according to claim 7, in which D-ribose allows skin elasticity to be improved.

9. Use according to claim 8, in which the D-ribose allows the freshness of the skin to be improved.

10. Use according to claim 7, in which the D-ribose has a degree of purity of at least 98% (HPLC).

11. Use according to claim 7, in which the D-ribose is present in a cosmetic composition at a concentration of 0.01 to 10% by weight with respect to the total weight of the cosmetic composition.

12. Use according to claim 11, in which the D-ribose is present in a cosmetic composition at a concentration of 0.1 to 1.5% by weight with respect to the total weight of the cosmetic composition.

13. Cosmetic composition comprising D-ribose at a concentration of 0.1 to 1.5% by weight with respect to the total weight of said composition
